(19) United States
(12) Patent Application Publication
(10) Pub. No.: US 2026/0341441 A1
(43) Pub. Date: Oct. 29, 2026

(54) ARTIFICIAL INTELLIGENCE SYSTEMS AND METHODS FOR DETERMINING EFFECTIVENESS OF THERAPEUTIC INTERVENTION SESSIONS

(71) Applicant: REGENTS OF THE UNIVERSITY OF MICHIGAN, Ann Arbor, MI (US)

(72) Inventors: Kathleen Marie Potempa, Ann Arbor, MI (US); Somayeh Mlaei, Ann Arbor, MI (US); Susan Butterworth, St. Petersburg, FL (US); Marna Flaherty-Robb, West Linn, OR (US); Stacia Potempa, Ann Arbor, MI (US)

(21) Appl. No.: 19/658,220

(22) Filed: Apr. 24, 2026

Related U.S. Application Data
(60) Provisional application No. 63/795,168, filed on Apr. 25, 2025.

(51) Int. Cl. G16H 15/00; G06F 21/62; G06F 40/295; G16H 20/70; G16H 50/20

(52) U.S. Cl. CPC G16H 15/00; G06F 21/6254; G06F 40/295; G16H 20/70; G16H 50/20

(57) ABSTRACT

Systems and methods are described for utilizing machine learning models (e.g., an LLM) to determine the effectiveness of cognitive or behavioral intervention sessions and/or modify provision of subsequent sessions. An example method involves: (i) obtaining a trained machine learning model trained to determine respective values of one or more effectiveness parameters, (ii), obtaining a transcript of a cognitive or behavioral intervention session, (iii) analyzing the transcript using the trained machine learning model, and (iv) synthesizing the determined respective values of the one or more effectiveness parameters for the cognitive or behavioral intervention session to generate an assessment outcome, wherein the assessment outcome includes a composite effectiveness score.

### CROSS-REFERENCE TO RELATED APPLICATIONS

This application claims the benefit of U.S. Provisional Application No. 63/795,168, filed Apr. 25, 2025, which is incorporated herein by reference in its entirety.

### FIELD OF THE INVENTION

The present disclosure relates to machine learning models, and more particularly, to utilizing trained machine learning models such as large language models (LLMs) to analyze and modify therapeutic interventions.

### BACKGROUND

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

In recent years, there has been an increasing recognition of the importance of effective communication between healthcare practitioners and clients, especially in the contexts of measuring competencies involved in cognitive or behavioral interventions, which may generally address behavior change, cognitive change, and/or treatment adherence in clients. Such cognitive or behavioral interventions may include, for example, motivational interviewing (MI) and/or other behavioral change therapies, but can include many other types of interventions on behalf of clients. Sessions in these interventions are widely used in healthcare, behavioral health, and health coaching settings because they provide an environment to help individuals identify and achieve their health goals. These sessions promote positive behavior change, help manage chronic conditions, and improves overall well-being through personalized plans, accountability, and motivational strategies, often focusing on lifestyle adjustments like diet, exercise, stress management, and sleep hygiene. A practitioner must possess a high level of skill and competency for cognitive or behavioral intervention sessions to be effective. Thus, there is immense value in ensuring that practitioners are provided with comprehensive training and ongoing quality assurance checks. The value of these sessions has been observed, as just an example, in behavioral change therapies such as motivational interviewing (MI), a client-centered counseling style for eliciting behavior change by helping clients to explore and resolve ambivalence.

Although practitioners are interested in determining and recording the effectiveness of cognitive or behavioral intervention sessions, it has been found that self-reported accounts by practitioners of the effectiveness of sessions are generally unreliable. As such, various tools have been developed and utilized by third parties to assess (or “code”) the competency or effectiveness of professionals in employing the techniques associated with these sessions. Different standardized, validated competency models may be provided for respective types of interventions. As just one example, the Motivational Interviewing Competency Assessment (MICA) evaluates the fidelity of MI principles in a practitioner's session with a client. However, in various cognitive or behavioral interventions, the traditional process of coding is labor-intensive, requiring considerable expertise and time, which can limit its reliability, accessibility and scalability.

Interest in leveraging technology to automate assessment (coding) for therapeutic strategies has grown, due to the need for efficient and timely assessment. While there are existing tools for measuring competencies, none have specifically addressed the automation of competency coding. This gap in the state of the art underscores the potential for innovative solutions that can streamline the assessment process, such as providing immediate feedback to practitioners and supporting the ongoing development of measuring competencies. Technological improvement in this area could significantly enhance the efficiency and scale of cognitive or behavioral intervention training and quality improvement initiatives within the healthcare, behavioral health, and health coaching settings.

Given the complexities involved in accurately assessing these techniques and the limitations of manual coding processes, the present disclosure identifies opportunities for improved platforms and technologies for solving the identified conventional problems.

### BRIEF SUMMARY OF THE INVENTION

In one aspect, a computing system includes: (1) a processor and (2) a memory that includes computer-executable instructions. The instructions, when executed, cause the computing system to (i) obtain a trained machine learning model trained to determine respective values of one or more effectiveness parameters of cognitive or behavioral intervention sessions, based on labeled training data indicative of respective values of the one or more effectiveness parameters for reference cognitive or behavioral intervention sessions; (ii) obtain a transcript of a cognitive or behavioral intervention session; (iii) analyze the transcript using the trained machine learning model to determine respective values of the one or more effectiveness parameters for the cognitive or behavioral intervention session; and (iv) synthesize the determined respective values of the one or more effectiveness parameters for the cognitive or behavioral intervention session to generate an assessment outcome, wherein the assessment outcome includes a composite effectiveness score.

In another aspect, a method includes: (1) obtaining, via one or more processors, a trained machine learning model trained to determine respective values of one or more effectiveness parameters of cognitive or behavioral intervention sessions, based on labeled training data indicative of respective values of the one or more effectiveness parameters for reference cognitive or behavioral intervention sessions; (2) obtaining, via the one or more processors, a transcript of a cognitive or behavioral intervention session; (3) analyzing, via the one or more processors, the transcript using the trained machine learning model to determine respective values of the one or more effectiveness parameters for the cognitive or behavioral intervention session; and (4) synthesizing, via the one or more processors, the determined respective values of the one or more effectiveness parameters for the cognitive or behavioral intervention session to generate an assessment outcome, wherein the assessment outcome includes a composite effectiveness score.

In yet another aspect, a computer-readable medium includes instructions that, when executed by a computing system comprising a processor, cause the computing system to perform operations including: (1) obtaining a trained machine learning model trained to determine respective values of one or more effectiveness parameters of cognitive or behavioral intervention sessions, based on labeled training data indicative of respective values of the one or more effectiveness parameters for reference cognitive or behavioral intervention sessions; (2) obtaining a transcript of a cognitive or behavioral intervention session; (3) analyzing the transcript using the trained machine learning model to determine respective values of the one or more effectiveness parameters for the cognitive or behavioral intervention session; and (4) synthesizing the determined respective values of the one or more effectiveness parameters for the cognitive or behavioral intervention session to generate an assessment outcome, wherein the assessment outcome includes a composite effectiveness score.

Advantages will become more apparent to those of ordinary skill in the art from the following description of the preferred embodiments which have been shown and described by way of illustration. As will be realized, the present embodiments may be capable of other and different embodiments, and their details are capable of modification in various respects. Accordingly, the drawings and description are to be regarded as illustrative in nature and not as restrictive.

### BRIEF DESCRIPTION OF THE DRAWINGS

The figures described below depict various aspects of the system and methods disclosed herein. It should be understood that each figure depicts an embodiment of a particular aspect of the disclosed system and methods, and that each of the figures is intended to accord with a possible embodiment thereof.

There are shown in the drawings arrangements which are presently discussed, it being understood, however, that the present embodiments are not limited to the precise arrangements and instrumentalities shown, wherein:

FIG. 1 illustrates an example computing environment for determining the effectiveness of cognitive or behavioral intervention sessions based on a machine learning model.

FIG. 2A illustrates an example architecture for evaluating the effectiveness of cognitive or behavioral intervention session techniques via a first evaluation model.

FIG. 2B illustrates another example architecture for evaluating the effectiveness of cognitive or behavioral intervention session techniques via a second evaluation model.

FIG. 2C illustrates still another example architecture for evaluating the effectiveness of cognitive or behavioral intervention session techniques via a third evaluation model.

FIG. 3 illustrates an exemplary flow diagram for a method for determining the effectiveness of cognitive or behavioral intervention sessions.

FIG. 4 illustrates another exemplary flow diagram of method elements associated with determining the effectiveness of cognitive or behavioral intervention sessions.

### DETAILED DESCRIPTION

The present techniques relate to the development and implementation of computing systems and methods for automating the assessment of various competencies associated with cognitive or behavioral intervention sessions, through the use of advanced machine learning and artificial intelligence technologies. The systems and methods herein address the need for efficient, accurate, and scalable evaluation of multiple types of professionals' abilities in cognitive or behavioral interventions, various examples of which use communication techniques useful for facilitating cognitive change, behavioral change, and/or treatment adherence in clients. The disclosed systems and methods enhance the process of assessing cognitive or behavioral intervention competencies by improving the traditionally manual, time-consuming, and resource-intensive task of coding and evaluating cognitive or behavioral intervention sessions. By leveraging a trained machine learning model, the systems and methods obtain and analyze transcripts of respective sessions to determine the effectiveness parameters and effectiveness sub-parameters of these sessions. This approach streamlines the assessment process and provides a foundation for generating reliable and actionable insights to improve future sessions, not limited to a singular focus, strategy, or skill set.

One of the improvements introduced by the present techniques is the ability to process and analyze cognitive or behavioral intervention session transcripts. This improvement is achieved at least through a trained machine learning model designed to identify and evaluate various effectiveness parameters and effectiveness sub-parameters of cognitive or behavioral intervention sessions. By analyzing these transcripts, the systems and methods herein can determine values for these parameters, which are then synthesized to generate a composite effectiveness score. The composite effectiveness score offers a quantified measure of the session's effectiveness, providing practitioners with a valuable tool for assessing and enhancing their approach to cognitive or behavioral intervention sessions.

Furthermore, the present techniques incorporate features such as speaker diarization processes and anonymization of audio data before analysis. The speaker diarization process identifies and separates contributions from different speakers within a cognitive or behavioral intervention session transcript. The speaker diarization capability ensures that the analysis is accurately attributed to the correct participants, enhancing the reliability of the cognitive or behavioral intervention session transcript and effectiveness assessment. Additionally, the anonymization of audio data prior to analysis enables the system to maintain client confidentiality and privacy.

Another key improvement via the techniques herein is the ability to update the trained machine learning model based on the determined effectiveness parameters in accordance with evaluation models of multiple different validated and standardized assessment tools. The ability to update the trained machine learning model enables the model to learn and adapt over time, increasing its accuracy and reliability in assessing sessions. Moreover, the systems and methods herein can generate detailed reports and recommendations to clients and/or practitioners based on the analysis, offering practitioners actionable insights to improve their practice and tailor interview strategies more effectively to individual client needs, and/or offering clients insights to improve their receipt of cognitive or behavioral interventions.

In summary, the present techniques represent a significant advancement in the field of behavioral health, healthcare, and health coaching. By enhancing the analysis of cognitive or behavioral intervention session transcripts using machine learning, the system offers a scalable, efficient, and privacy-conscious method for evaluating and enhancing the effectiveness of various different types of cognitive or behavioral interventions using evidence-based strategies. These improvements contribute significantly to use cases spanning various industries.

One of the core components of this method is the utilization of a machine learning model, which has been fine-tuned specifically for the task of scoring sessions based on the relevant criteria for an assigned cognitive/behavioral intervention strategy, skill set, or characteristic of a validated standardized reference tool (e.g., motivational interviewing therapy competency models such as Motivational Interviewing Competency Assessment (MICA)). Assessment criteria vary depending on the assessment tool being implemented. Various tools are considered by this method, and are not limited to but may include exemplary MI tools such as the MICA, the Nurse Health Coach Competency Assessment (NHCCA), Indicators of Health Behavior Change (IHBC), and/or other similar tools. This approach leverages the vast capabilities of generative AI models, enriched with domain-specific knowledge and data, to accurately interpret and evaluate the nuances of sessions. The AI model's ability to process and analyze text-based transcripts of these sessions allows for the rapid generation of MI scores, offering immediate feedback to practitioners and facilitating a more dynamic and responsive learning environment.

The systems and methods herein further encompass the integration of expert knowledge and guidance into the AI model's training process. This is achieved by incorporating detailed instructions, examples, and criteria derived from various sources, such as coding manuals for standardized, validated assessment tools (e.g., the MICA coding manual). Such integration ensures that the AI model is well-informed and aligned with the established standards and expectations of competencies. By doing so, the systems and methods herein not only automate and improves the scoring process, but also ensure that the evaluations are grounded in the rich context and depth of understanding that expert human coders conventionally bring to the task, in addition to the further benefits particular to the machine learning implementations described herein.

Moreover, the systems and methods herein introduce a user-friendly interface designed to streamline the entire process from session recording to feedback delivery. This interface facilitates the secure and efficient management of audio files, their conversion to text transcripts, and the subsequent processing by the AI model. The interface provides a seamless experience for both practitioners seeking evaluation and administrators overseeing the quality improvement efforts within their organizations. The interface's design prioritizes privacy, ease of use, and accessibility, making the advanced capabilities of the AI-based assessment method readily available to a wide range of users.

One or more machine learning models may be created and trained based upon example data (e.g., “training data”) inputs or data (which may be termed “features” and “labels”) to make valid and reliable predictions for new inputs, e.g., inputs in the form of cognitive or behavioral intervention session transcripts. In supervised machine learning, a machine learning program operating on a server, computing device, or otherwise processor(s), may be provided with example inputs (e.g., “features”) and their associated, or observed, outputs (e.g., “labels”) in order for the machine learning program or algorithm to determine or discover rules, relationships, patterns, or otherwise machine learning “models” that map such inputs (e.g., “features”) to the outputs (e.g., labels), for example, by determining and/or assigning weights or other metrics to the model across its various feature categories. Such rules, relationships, or otherwise models may then be provided subsequent inputs in order for the model, executing on the server, computing device, or otherwise processor(s), to predict, based upon the discovered rules, relationships, or model, an expected output.

In unsupervised machine learning, the server, computing device, or otherwise processor(s), may be required to find its own structure in unlabeled example inputs, where, for example multiple training iterations are executed by the server, computing device, or otherwise processor(s) to train multiple generations of models until a satisfactory model, e.g., a model that provides sufficient prediction accuracy when given test level or production level data or inputs, is generated.

The techniques herein may use one or both of such supervised or unsupervised machine learning techniques. For example, in some embodiments, supervised machine learning techniques may include training and/or applying a supervised machine learning model to produce an output score and/or other characterization of a cognitive or behavioral intervention session based on input data (e.g., a transcript of the session). Additionally or alternatively, in embodiments, unsupervised machine learning techniques may include training and/or applying an unsupervised machine learning model such as a classification model by one or more properties, e.g., to compare cognitive or behavioral intervention sessions against each other and/or to generate discrete scores or other characterizations of each classified group of sessions.

In any case, the present techniques may include training and/or applying machine learning models, including large language models (LLMs), by processing datasets to improve model parameters for specific tasks. LLMs are AI systems trained on extensive corpora of text data to understand and generate human-like text. These models, such as GPT (Generative Pre-trained Transformer) and other such models, leverage deep learning techniques to process and produce text that can mimic human writing styles, answer questions, summarize information, and more. The training process involves the use of extensive preprocessed datasets that may include textual, numerical, or multimodal data. The systems and methods herein may employ techniques such as gradient descent to adjust model weights iteratively. For example, during training, data is divided into batches, and each batch is passed through the model to compute predictions. The loss between predictions and ground truth labels is calculated using a loss function, and gradients are backpropagated through the model to update the model's parameters. The training process is repeated over multiple epochs until convergence criteria are met.

In some embodiments, the systems and methods herein may additionally include mechanisms for fine-tuning pre-trained LLMs on domain-specific datasets to improve task performance. Fine-tuning involves initializing the model with weights from a pre-trained state and then training the model further on a smaller, curated dataset specific to the desired application. Regularization techniques such as dropout and weight decay are employed during fine-tuning to prevent overfitting and maintain generalization. Additionally, optimization algorithms such as Adam or RMSProp may be used to adapt the learning rate dynamically, ensuring stability and convergence throughout training.

In some embodiments, to enhance training efficiency, the machine learning techniques herein distribute training across multiple computational nodes. Each node processes a subset of the data in parallel, and updates to model parameters are synchronized across nodes using methods such as gradient averaging or parameter server architectures. The distributed training approach enables the systems and methods herein to handle large datasets and high-dimensional models efficiently. The systems and methods herein may also incorporate mixed-precision training, where lower-precision arithmetic is used for certain computations to reduce memory usage and improve computational speed without compromising model accuracy.

In some embodiments, the training process for LLMs may include the use of specialized tokenization methods to preprocess textual input. Tokenization involves converting raw text into tokens that represent words, subwords, or characters, depending on the model architecture. The tokens are mapped to unique identifiers and fed into the model's embedding layer. Positional encodings are added to the embeddings to capture the order of tokens within the input sequence. Attention mechanisms, such as those used in transformer architectures, process these embeddings to capture long-range dependencies in the text, ensuring the model learns contextually relevant patterns.

In some embodiments, the disclosed systems and methods employ a validation phase during training to evaluate model performance on unseen data. At regular intervals, a separate validation dataset is used to compute metrics such as accuracy, loss, or F1 score. These metrics guide the adjustment of hyperparameters such as learning rate, batch size, and dropout rate. Early stopping mechanisms halt training when validation performance plateaus or deteriorates, preventing overfitting. The systems and methods may also include functionality for saving checkpoints of model parameters at regular intervals, allowing training to resume from the latest checkpoint in case of interruption.

These training techniques may be applied to develop machine learning models, particularly LLMs, that can perform tasks such as natural language understanding, text generation, and context-aware reasoning. By leveraging large-scale data, efficient training algorithms, and advanced architectures, the disclosed systems and methods improve LLM performance for a wide range of applications.

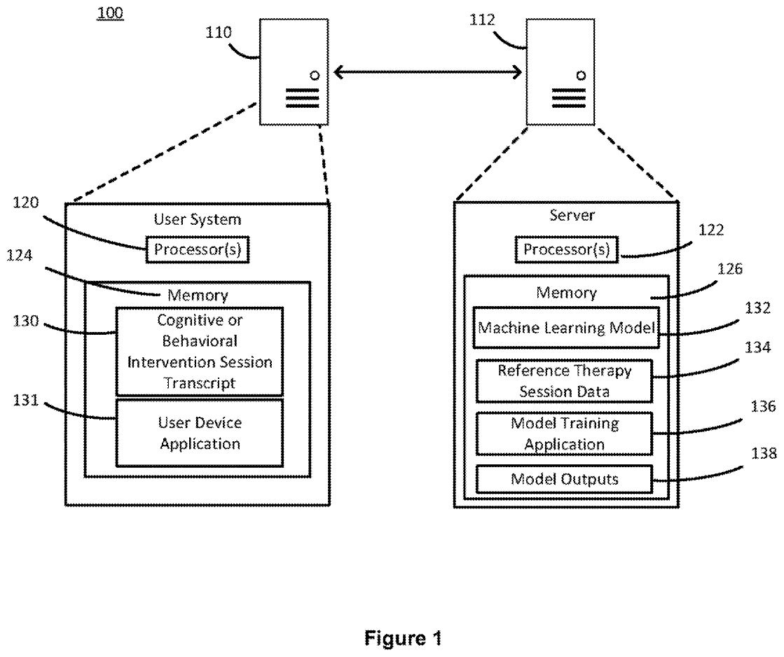

Referring now to the drawings, FIG. 1 depicts an exemplary computing environment 100 for determining the effectiveness of cognitive or behavioral intervention sessions (e.g., motivational interviewing sessions, other behavioral change sessions, and/or still other interventions), according to some embodiments. The computing environment 100 includes a user system 110 and a server 112. The computing environment 100 illustrated in FIG. 1 may include both hardware and software applications, as well as various data communications channels for communicating data between the various hardware and software components, as is described below. The computing environment 100 illustrated in FIG. 1 further includes various components and channels for communication and data flow, as is described below.

The user system 110 may, for example, include a desktop computer, laptop computer, smartphone, tablet, wearable computing device, and/or other client computing device(s). The user system 110 contains processor(s) 120 a memory 124, which may further include a cognitive or behavioral intervention session transcript 130 (e.g., MICA or other behavioral change therapy transcript) and user device application 131. In some embodiments, the user system 110 includes storage capabilities for various data types that are utilized in or during operation. The memory 124 of the user system 110 may include one or more forms of volatile and/or non-volatile, fixed and/or removable memory, such as read-only memory (ROM), electronic programmable read-only memory (EPROM), random access memory (RAM), erasable electronic programmable read-only memory (EEPROM), and/or other hard drives, flash memory, MicroSD cards, and others. The memory 124 stores a user device application 131. In some embodiments, the memory 124 of the user system 110 may also store a web browser via which a user can access a service provided by the organization that owns the user system 110 or via which a user can operate or access a user device application 131 that accesses the user system 110. In further embodiments, the user device application 131 may collect therapy session data from the cognitive or behavioral intervention session transcript 130. In even further embodiments, the user device application 131 can display results of the processing of the cognitive or behavioral intervention session transcript 130.

The server 112 includes processor(s) 122 and a memory 126. The memory 126 of the server 112 may include a machine learning model 132, reference therapy session data 134, a model training application 136, and model outputs 138. In some embodiments, the server 112 uses the machine learning model 132 trained by model training application 136 using reference therapy session data 134 to generate model outputs 138. In some embodiments, the server 112 stores the cognitive or behavioral intervention session transcript 130 (additionally or alternatively to the cognitive or behavioral intervention session transcript 130 being stored at the user system 110).

In some embodiments, the server 112 may be implemented as cloud-based servers, such as a cloud-based computing platform. For example, the server 112 may be any one or more cloud-based platform(s) such as MICROSOFT AZURE, AMAZON AWS, or the like. Either or both user system 110 and server 112 can further include a user interface, for example to display alerts or other indications as described later herein. The user interfaces may be provided in whole or in part on a display monitor (e.g., LCD screen, touch screen, or any other type of display), and can incorporate an integrated or separate sound system.

In some embodiments, the machine learning model 132 generates vector embeddings to represent entities, concepts, or objects in a high-dimensional numerical space. Such embeddings may be created by processing input data using techniques such as word2vec, GloVe, or transformer-based models. The input data may include textual descriptions, contextual relationships, or domain-specific metadata. Each entity is mapped to a unique embedding, encapsulating the entity's features and relationships within the dataset. The embeddings are stored in a structured vector space that allows for efficient similarity computations using methods such as cosine similarity or Euclidean distance.

In some embodiments, the generated vector embeddings may be stored in a database that supports scalable retrieval and computation. The system may enable queries to identify embeddings that are similar based on a spatial proximity in the vector space. Proximity measurements are used to infer relationships, clusters, or patterns among entities. The embeddings may be updated periodically through a feedback mechanism that incorporates data from user interactions and performance metrics. The embeddings update process ensures the embeddings remain aligned with evolving datasets and tasks. The system also provides tools to query and analyze the embedding space for trends or outliers.

In some embodiments, the machine learning model 132 integrates knowledge graphs to represent structured relationships between entities. Each knowledge graph consists of nodes, which correspond to entities, and edges, which define relationships between these nodes. The graph structure captures both explicit relationships and inferred associations. Node attributes and edge properties provide additional contextual information about entities and corresponding connections. The machine learning model 132 may support the dynamic creation, update, and management of the knowledge graph as new data is ingested or relationships are modified.

In some embodiments, the machine learning model 132 integrates vector embeddings with the knowledge graph to enhance the utility for queries and reasoning. Each node in the graph is associated with a corresponding vector embedding, which is generated or updated based on the attributes of the node and the graph structure. Embedding generation methods include graph convolutional networks or graph attention networks, which incorporate both local and global structural properties of the graph. The machine learning model 132 also allow embeddings to be generated for unstructured data, enabling incorporation into the knowledge graph as new nodes or relationships. The integration of vector embeddings and knowledge graphs support efficient traversal, reasoning, and querying operations using both the discrete graph structure and the continuous vector space.

The integration of vector embeddings and knowledge graphs provides a unified approach to managing and querying complex datasets. The vector embeddings represent entities in a continuous space that captures semantic similarity, while the knowledge graph provides a discrete structure that captures explicit relationships. The embeddings enhance the graph by enabling similarity-based reasoning, while the graph enriches the embeddings with structured relational context. Together, the components allow for efficient knowledge representation and retrieval in applications requiring both structured and unstructured data.

In some embodiments, the machine learning model 132 may be called and/or enhanced through particularly engineered prompts. In some such embodiments, prompt engineering may involve the strategic formulation and structuring of prompts or queries to effectively interact with and guide LLMs towards generating desired outputs. Prompt engineering leverages the understanding of how LLMs interpret and process natural language inputs to craft prompts that can elicit specific types of responses or information. By carefully designing these prompts, users can enhance the relevance, accuracy, and utility of the responses generated by LLMs. Prompt engineering may therefore enable more efficient and targeted extraction of knowledge from vast datasets. For example, in embodiments, described herein, prompts to an LLM include instructions prepared by cognitive or behavioral intervention experts (e.g., experts in motivational interviewing or MICA assessments) regarding how the LLM is to assess particular aspects of assessment of a cognitive or behavioral intervention session (e.g., particular parameters or sub-parameters in a standardized, validated assessment such as a MICA or other behavioral change session, as will be described further herein).

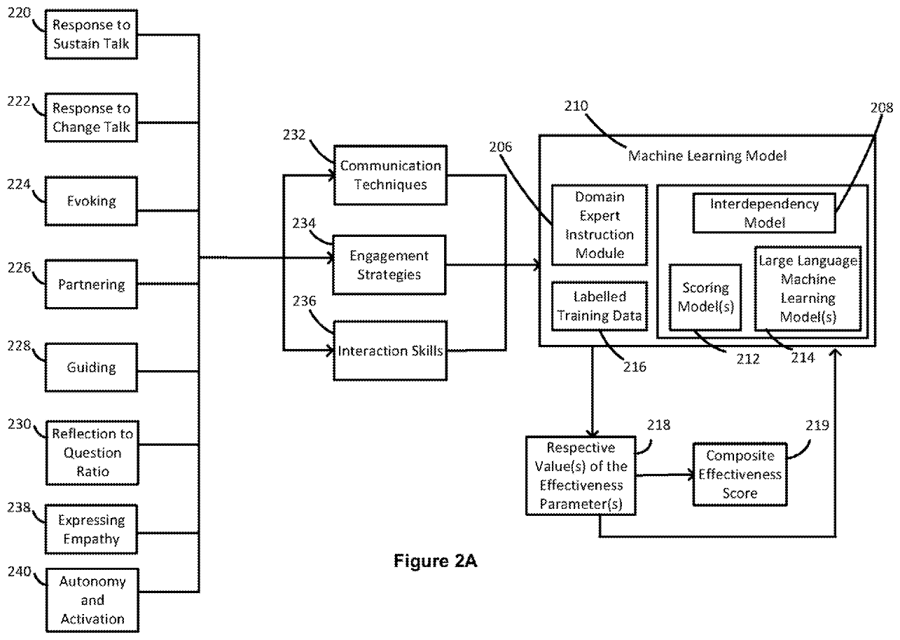

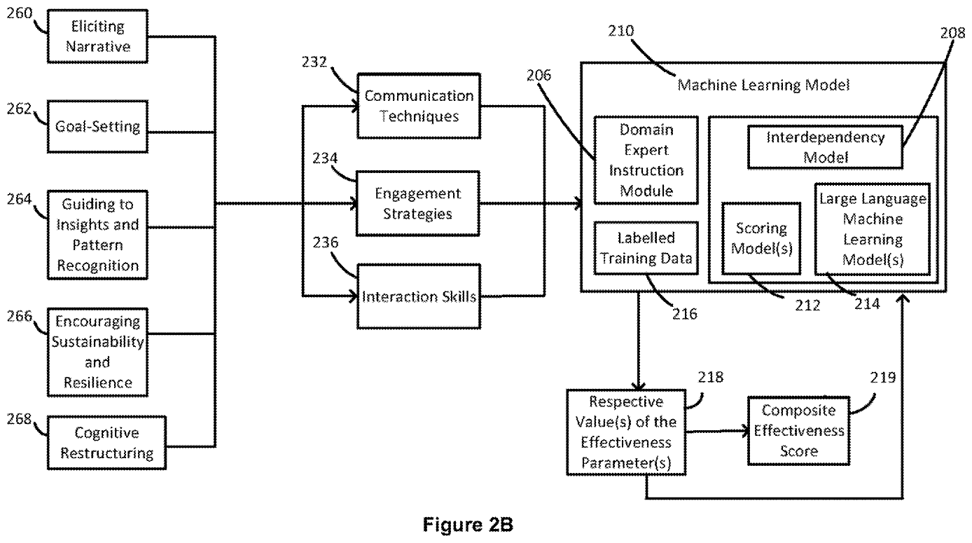

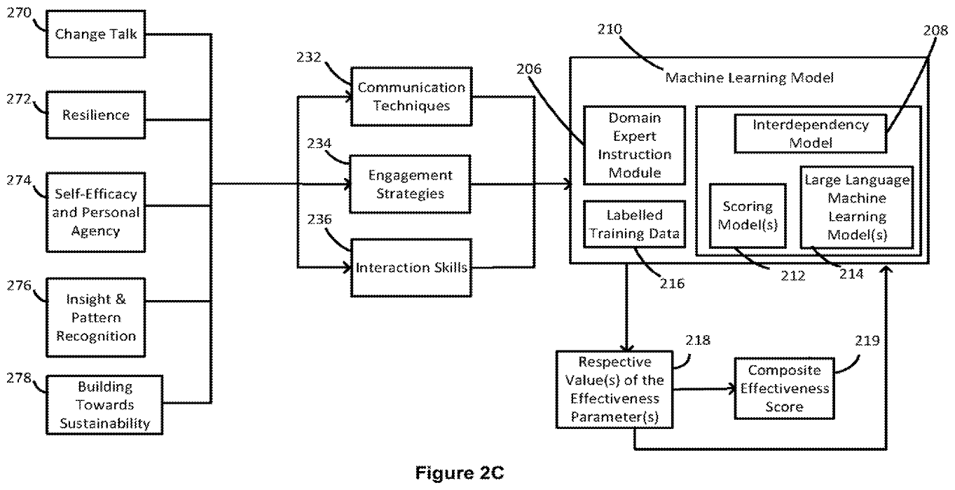

FIG. 2A illustrates a first exemplary architecture for evaluating the effectiveness of cognitive or behavioral intervention sessions within a computing environment, according to some embodiments. In particular, as depicted through certain elements in FIG. 2A, the cognitive or behavioral intervention session may include a MICA therapy session, although it should be appreciated from this disclosure that other types of interventions are envisioned by this disclosure. In various embodiments, the architecture of FIG. 2A is implemented via suitable components of the computing environment 100 of FIG. 1. This architecture is designed such that effectiveness parameters representing different aspects of motivational interviewing (or, in alternative implementations, aspects of a standardized, validated model of another type of cognitive or behavioral change intervention) feed into a central machine learning model (e.g., machine learning model 132 of FIG. 1), with these components generating scores that reflect the effectiveness of the motivational interviewing therapy sessions. In some embodiments, a number of distinct effectiveness parameters may be used to embody specific motivational interviewing communication aspects. Depending on the embodiment, the effectiveness parameters may include a communication techniques effectiveness parameter 232, an engagement strategies effectiveness parameter 234, and interaction skills effectiveness parameter 236. In some embodiments, a number of distinct effectiveness sub-parameters may be used, each with a role in evaluating the specific communicational aspects associated with the effectiveness parameters. Depending on the embodiment, the sub-parameters may include a response to sustain talk effectiveness sub-parameter 220, a response to change talk effectiveness sub-parameter 222, an evoking effectiveness sub-parameter 224, a partnering effectiveness sub-parameter 226, a guiding effectiveness sub-parameter 228, a reflection to question ratio effectiveness sub-parameter 230, an expressing empathy sub-parameter 238 and a supporting autonomy and activation sub-parameter 240. The various data classified as effectiveness parameters and effectiveness sub-parameters as a whole are considered composite effectiveness data. The processed composite effectiveness data is then synthesized to generate a composite effectiveness score.

Within the central machine learning model 210, there are five model components: one or more scoring models 212, one or more large language machine learning models 214, an interdependency model 208, a domain expert instruction module 206, and labelled training data 216, each with a distinct function in the evaluation process.

In some embodiments, the interdependency model 208 may govern the training of the various machine learning models. For example, in some embodiments the interdependency model 208 can train a model (or a particular portion of the model) based on a previously trained model (or a particular portion of the model previously trained). For example, training of the model to assess a first one or more of the effectiveness sub-parameters of FIG. 2A (e.g., the expressing empathy sub-parameter 238) may depend on the model (or portion thereof) trained to assess a second, different one or more of the effectiveness sub-parameters of FIG. 2A (e.g., the reflection to question ratio effectiveness sub-parameter 230). Thus, in some embodiments, the interdependency model 208 control the training process for components of the central machine learning model 210, or portions of the various components of the central machine learning model 210.

In some embodiments, the domain expert instruction module 206 may influence training of the various models in the central machine learning model 210 by receiving instructions submitted from a cognitive or behavioral intervention expert (e.g., in the particular implementation depicted in FIG. 2A, a motivational interviewing domain expert). In some embodiments, the cognitive or behavioral intervention expert can communicate with the domain expert instruction module in a user interface to direct the domain expert instruction module to influence the training of the various models in the central machine learning model 210 based on the communication.

In some embodiments, the scoring models 212 align cognitive or behavioral intervention techniques (e.g., in this example, motivational interviewing techniques) with existing effectiveness benchmarks, such as the effectiveness parameters and effectiveness sub-parameters. The scoring models 212 identify areas where current techniques may need adjustment.

In some embodiments, the large language machine learning models 214 leverage LLMs, prompt engineering, and/or vector embeddings to process unstructured data from diverse sources, including text, tables, and diagrams. Such features may enable these models to provide valuable insights into how cognitive or behavioral intervention techniques (e.g., motivational interviewing techniques) can be effectively evaluated and enhanced.

In some embodiments, the labelled training data 216 is tailored to individual cognitive or behavioral intervention (e.g., motivational interviewing) effectiveness parameters and effectiveness sub-parameters. In further embodiments, the labelled training data 216 trains the central machine learning model 210 based on feedback and learning from reference cognitive or behavioral intervention session data (e.g., motivational interviewing session data).

In some embodiments, the central machine learning model 210 receives the transcript of the cognitive or behavioral intervention session (e.g., the motivational interview therapy session). The trained central machine learning model 210, using the scoring models 212 and the large language machine learning models 214, processes the transcript of the session to determine respective values of the effectiveness parameters 218, which may include respective values of the effectiveness sub parameters. The respective values of the effectiveness parameters 218 are then synthesized by the central machine learning model 210 to generate a composite effectiveness score 219. The respective values of the effectiveness parameters 218 may then be received and processed by the central machine learning model 210 to further train and update the central machine learning model 210.

FIG. 2A thus presents a high-level architecture for a complex system designed to evaluate various effectiveness parameters and effectiveness sub-parameters of cognitive or behavioral intervention sessions (e.g., in particular embodiments, motivational interviewing therapy sessions) through specialized components. The system then further processes and aligns these effectiveness parameters and effectiveness sub-parameters within a central machine learning model, ensuring that the evaluation system continuously updates and is informed by feedback learning. Where motivational interviewing therapy sessions are described in the architecture of FIG. 2A, it should be appreciated that the architecture of FIG. 2A may be adapted to another type(s) of cognitive or behavioral intervention session and/or framework for assessing said therapy sessions, e.g., by replacing described parameters in accordance with the other type(s) of behavior change therapy session or assessment framework. For example, a cognitive or behavioral intervention session could be a motivational interviewing therapy session, client education session, chronic condition management session, addiction therapy session, psychiatric therapy session, or speech therapy session.

FIG. 2B illustrates another exemplary architecture similar to FIG. 2A for evaluating effectiveness of cognitive or behavioral intervention sessions according to some embodiments. In the particular case of FIG. 2B, the architecture uses using NHCCA tools within a computing environment analogous to that described with respect to FIG. 2A. In various embodiments, the architecture of FIG. 2B is implemented via suitable components of the computing environment 100 of FIG. 1. This architecture is designed such that effectiveness parameters representing different aspects of the NHCCA tools feed into a central machine learning model (e.g., machine learning model 132 of FIG. 1), with these components generating scores that reflect the effectiveness of the sessions. In some embodiments, a number of distinct effectiveness parameters may be used to embody specific aspects of sessions. Depending on the embodiment, the effectiveness parameters may include a communication techniques effectiveness parameter 232, an engagement strategies effectiveness parameter 234, and interaction skills effectiveness parameter 236. In some embodiments, a number of distinct effectiveness sub-parameters may be used, each with a role in evaluating the specific communicational aspects associated with the effectiveness parameters. Depending on the embodiment, the sub-parameters may include an eliciting narrative sub-parameter 260, a goal-setting sub-parameter 262, a guiding to insights and pattern recognition sub-parameter 264, an encouraging sustainability and resilience sub-parameter 266, and a cognitive restructuring sub-parameter 268. The various data classified as effectiveness parameters and effectiveness sub-parameters as a whole are considered composite effectiveness data. The processed composite effectiveness data is then synthesized to generate a composite effectiveness score 219.

FIG. 2C illustrates still another exemplary architecture similar to FIG. 2A for evaluating effectiveness using IHBC tools within a computing environment, according to some embodiments. In various embodiments, the architecture of FIG. 2C is implemented via suitable components of the computing environment 100 of FIG. 1. This architecture is designed such that effectiveness parameters representing different aspects of sessions feed into a central machine learning model (e.g., machine learning model 132 of FIG. 1), with these components generating scores that reflect the effectiveness of the sessions. In some embodiments, a number of distinct effectiveness parameters may be used to embody specific aspects of sessions. Depending on the embodiment, the effectiveness parameters may include a communication techniques effectiveness parameter 232, an engagement strategies effectiveness parameter 234, and interaction skills effectiveness parameter 236. In some embodiments, a number of distinct effectiveness sub-parameters may be used, each with a role in evaluating the specific communicational aspects associated with the effectiveness parameters. Depending on the embodiment, the sub-parameters may include a change talk sub-parameter 270, a resilience sub-parameter 272, a self-efficacy and personal agency sub-parameter 274, an insight and pattern recognition sub-parameter 276, and a building towards sustainability sub-parameter 278. The various data classified as effectiveness parameters and effectiveness sub-parameters as a whole are considered composite effectiveness data. The processed composite effectiveness data is then synthesized to generate a composite effectiveness score 219.

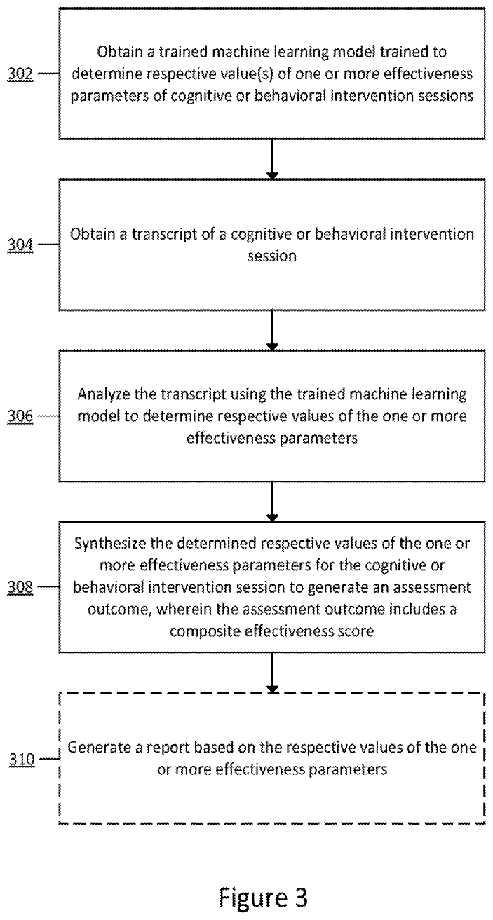

FIG. 3 presents an exemplary flow diagram of a method for determining the effectiveness of cognitive or behavioral intervention sessions (e.g., motivational interviewing sessions). The method may be implemented by a computing environment 100, a component of the computing environment 100, a computing device communicating with the computing environment 100, and/or any other such device as described herein. It will be understood that additional, fewer, and/or alternate components may be used to implement the example method, and/or that the method may include more or fewer steps than shown (and/or in a different order than shown).

In some embodiments, at step 302, a computing device obtains a trained machine learning model specifically trained to determine respective value(s) of one or more effectiveness parameters of cognitive or behavioral intervention sessions (e.g., motivational interviewing sessions). This step underscores the utilization of advanced machine learning techniques to assess and quantify the impact of the nuances of intervention activities (e.g., therapeutic conversations). In some embodiments, the step 302 includes training the machine learning model, which may utilize supervised and/or unsupervised machine learning techniques.

In some embodiments, at step 304, the computing device obtains a transcript of a cognitive or behavioral intervention session (e.g., a motivational interview therapy session). The transcript provides a detailed account of the interactions and conversation between the interviewer and the interviewee. In some embodiments, step 304 includes converting an audio transcript of the cognitive or behavioral intervention session to a text transcript. In some embodiments, the method also includes a speaker diarization process, to identify and separate respective contributions from a plurality of different speakers indicated by the transcript. The diarization process can for example include identifying each one of the plurality of speakers as an interviewer (e.g., practitioner) or interviewee (e.g., client).

In some embodiments, at step 306, the computing device analyzes the transcript using the trained machine learning model to determine respective values of the one or more effectiveness parameters (e.g., MICA parameters). This analysis applies the trained machine learning model to the real-world data collected in step 304 to extract quantifiable insights associated with the effectiveness of the cognitive or behavioral intervention techniques employed during the session. In some embodiments, the computing device anonymizes audio data comprising the transcript before providing the audio data to the trained machine learning model.

In some embodiments, at step 308, the computing device synthesizes the determined respective values of the one or more effectiveness parameters for the cognitive or behavioral intervention session to generate an assessment outcome. Depending on the criteria of the session being evaluated, the assessment outcome may include multiple global scores and/or a composite effectiveness score (e.g., a composite MICA score, in cases where the cognitive or behavioral intervention includes motivational interviewing), which provides a comprehensive view of the cognitive or behavioral intervention session's impact. This step translates the analytical findings into actionable insights that can guide interviewers in refining their approaches.

In some embodiments, at step 310, the computing device optionally generates a report based on the respective values of the one or more effectiveness parameters. The generated report may offer a comprehensive overview of the cognitive or behavioral intervention session's effectiveness, including recommendations for future sessions and highlighting areas of particular success or need for improvement.

In some embodiments, the method of FIG. 3 can further include generating a report based on the respective values of the one or more effectiveness parameters for the cognitive or behavioral intervention session, and/or updating the trained machine learning model based on the determined respective values of the one or more effectiveness parameters for the cognitive or behavioral intervention session. Still additionally or alternatively, in embodiments, the method of FIG. 3 can include generating a recommendation for a subsequent cognitive or behavioral intervention session based on the determined values of the one or more effectiveness parameters, generating a performance score of a practitioner (e.g., therapist) from the cognitive or behavioral intervention session (e.g., administrator of the therapy session) based on the determined values of the one or more effectiveness parameters, and/or generating a recommendation of a practitioner for a client based on the generated performance score of the practitioner by whom the analyzed cognitive or behavioral intervention session was administered (e.g., a same or different practitioner, with the recommendation being presented for example in the form of a graphical user interface or other interface at a device of the client receiving the intervention).

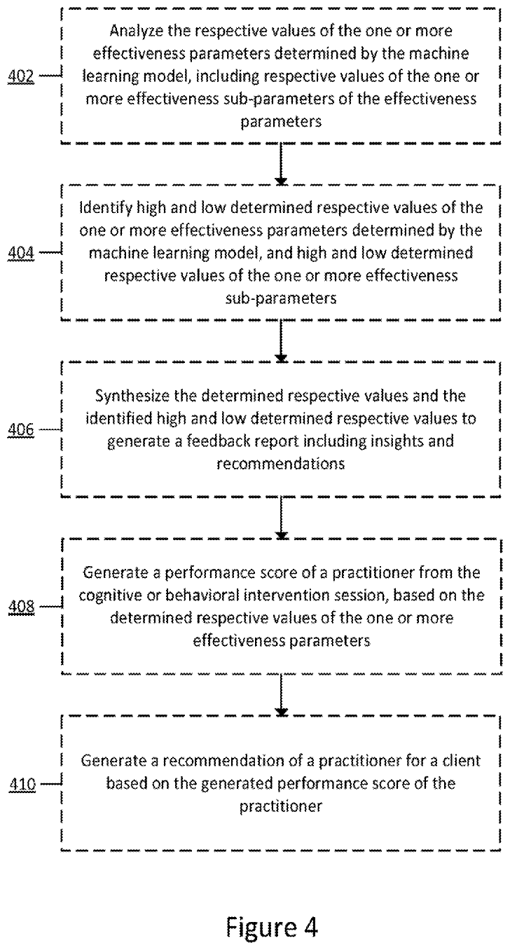

FIG. 4 presents an exemplary flow diagram of method elements associated with determining the effectiveness of cognitive or behavioral intervention sessions (e.g., motivational interviewing therapies). In various embodiments, one or more actions of the method of FIG. 4 follow execution of at least a portion of the method of FIG. 3. The method may be implemented by a machine learning model, a component of the machine learning model, a computing device communicating with the machine learning model, and/or any other such device as described herein. It will be understood that additional, fewer, and/or alternate components may be used to implement the example method, and/or that the method may include more or fewer steps than shown (and/or in a different order than shown).

In some embodiments, at step 402, a computing device may analyze the respective values of one or more effectiveness parameters determined by a machine learning model with respect to a cognitive or behavioral intervention session (e.g., as determined via step 306 of FIG. 3, with respect to a motivational interviewing or various other cognitive or behavioral intervention sessions). This includes analyzing respective values of the one or more effectiveness sub-parameters of the effectiveness parameters.

In some embodiments, at step 404, the computing device identifies high and low determined respective values of the one or more effectiveness parameters determined by the machine learning model, with high values indicating the effectiveness parameter was most effective and low values indicating the effectiveness parameter was less effective. Additionally or alternatively, the step 404 may identify high and low determined respective values of the one or more effectiveness sub-parameters.

In some embodiments, at step 406, the computing device synthesizes the determined respective values and the identified high and low determined respective values to generate a feedback report. This feedback report can include insights and/or recommendations based on the analysis conducted in the previous steps (e.g., to modify subsequent cognitive or behavioral intervention sessions via different techniques or sequences). The feedback report is a tool for practitioners to understand the effectiveness of their cognitive or behavioral interventions.

In some embodiments, at step 408, the computing device generates a performance score of a practitioner (e.g., therapist) from the cognitive or behavioral intervention session (e.g., a practitioner who administered the cognitive or behavioral intervention session to a client). This score is based on the determined respective values of the one or more effectiveness parameters. The performance score is a quantitative measure of a practitioner's effectiveness in delivering cognitive or behavioral interventions.

In some embodiments, at step 410, the computing device generates a recommendation of a practitioner for a client (e.g., a therapist recommendation) based on the generated performance score of the practitioner from the cognitive or behavioral intervention session. This step may involve matching clients with practitioners who have demonstrated high effectiveness for specific parameters in their cognitive or behavioral interventions that are most effective for a specific client.

### ASPECTS

The various embodiments described above can be combined to provide further embodiments. All U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

Aspects of the techniques described in the present disclosure may include any of the following aspects, either alone or in combination:
Aspect 1. A computing system comprising: a processor and a memory having stored thereon computer-executable instructions that, when executed, cause the computing system to: obtain a trained machine learning model trained to determine respective values of one or more effectiveness parameters of cognitive or behavioral intervention sessions, based on labeled training data indicative of respective values of the one or more effectiveness parameters for reference cognitive or behavioral intervention sessions; obtain a transcript of a cognitive or behavioral intervention session; analyze the transcript using the trained machine learning model to determine respective values of the one or more effectiveness parameters for the cognitive or behavioral intervention session; and synthesize the determined respective values of the one or more effectiveness parameters for the cognitive or behavioral intervention session to generate an assessment outcome, wherein the assessment outcome includes a composite effectiveness score.
Aspect 2. The computing system of aspect 1, wherein the trained machine learning model includes one or more of: 1) one or more scoring models, or 2) one or more large language machine learning models.
Aspect 3. The computing system of any of aspects 1-2, further comprising instructions that, when executed, cause the computing system to utilize a speaker diarization process to identify and separate respective contributions from a plurality of different speakers indicated by the transcript.
Aspect 4. The computing system of any of aspects 1-3, wherein the cognitive or behavioral intervention session is a motivational interview therapy session.
Aspect 5. The computing system of any of aspects 1-4, wherein the speaker diarization process includes identifying each respective one of the plurality of speakers as either an interviewer or an interviewee.
Aspect 6. The computing system of any of aspects 1-5, further comprising instructions that, when executed, cause the computing system to anonymize audio data before the analyzing using the trained machine learning model.
Aspect 7. The computing system of any of aspects 1-6, further comprising instructions that, when executed, cause the computing system to update the trained machine learning model based on the determined respective values of the one or more effectiveness parameters for the cognitive or behavioral intervention session.
Aspect 8. The computing system of any of aspects 1-7, further comprising instructions that, when executed, cause the computing system to generate a report based on the respective values of the one or more effectiveness parameters for the cognitive or behavioral intervention session.
Aspect 9. The computing system of any of aspects 1-8, wherein the one or more effectiveness parameters include one or more of: 1) communication techniques, 2) engagement strategies, or 3) interaction skills.
Aspect 10. The computing system of any of aspects 1-9, wherein the one or more effectiveness parameters are further comprised of one or more effectiveness sub-parameters, wherein the one or more effectiveness sub-parameters include one or more of: 1) evoking, 2) partnering, 3) guiding, 4) expressing empathy, 5) strategic response to sustain talk, 6) strategic response to change talk, 7) reflection to question ratio, and 8) supporting autonomy and activation.
Aspect 11. The computing system of any of aspects 1-10, wherein the computer-executable instructions, when executed, further cause the computing system to generate a therapy recommendation for a subsequent therapy session based on the determined values of the one or more effectiveness parameters.
Aspect 12. The computing system of any of aspects 1-11, wherein the computer-executable instructions, when executed, further cause the computing system to generate a performance score of a practitioner from the cognitive or behavioral intervention session, based on the determined values of the one or more effectiveness parameters.
Aspect 13. The computing system of any of aspects 1-12, wherein the computer-executable instructions, when executed, further cause the computing system to generate a recommendation of a practitioner for a client based on the generated performance score of the practitioner.
Aspect 14. A method comprising: obtaining, via one or more processors, a trained machine learning model trained to determine respective values of one or more effectiveness parameters of cognitive or behavioral intervention sessions, based on labeled training data indicative of respective values of the one or more effectiveness parameters for reference cognitive or behavioral intervention sessions; obtaining, via the one or more processors, a transcript of a cognitive or behavioral intervention session; analyzing, via the one or more processors, the transcript using the trained machine learning model to determine respective values of the one or more effectiveness parameters for the cognitive or behavioral intervention session; and synthesizing, via the one or more processors, the determined respective values of the one or more effectiveness parameters for the cognitive or behavioral intervention session to generate an assessment outcome, wherein the assessment outcome includes a composite effectiveness score.
Aspect 15. The method of aspect 14, wherein the trained machine learning model includes one or more of: 1) one or more scoring models, or 2) one or more large language machine learning models.
Aspect 16. The method of any of aspects 14-15, further comprising utilizing, via the one or more processors, a speaker diarization process to identify and separate respective contributions from a plurality of different speakers indicated by the transcript.
Aspect 17. The method of any of aspects 14-16, wherein the cognitive or behavioral intervention session is a motivational interview therapy session.
Aspect 18. The method of any of aspects 14-17, wherein the speaker diarization process includes identifying each respective one of the plurality of speakers as either an interviewer or an interviewee.
Aspect 19. The method of any of aspects 14-18, further comprising anonymizing, via the one or more processors, audio data before analyzing using the trained machine learning model.
Aspect 20. The method of any of aspects 14-19, further comprising updating, via the one or more processors, the trained machine learning model based on the determined respective values of the one or more effectiveness parameters for the cognitive or behavioral intervention session.
Aspect 21. The method of any of aspects 14-20, further comprising generating, via the one or more processors, a report based on the respective values of the one or more effectiveness parameters for the cognitive or behavioral intervention session.
Aspect 22. The method of any of aspects 14-21, wherein the one or more effectiveness parameters include one or more of: 1) communication techniques, 2) engagement strategies, or 3) interaction skills.
Aspect 23. The method of any of aspects 14-22, wherein the one or more effectiveness parameters are further comprised of one or more effectiveness sub-parameters, wherein the one or more effectiveness sub-parameters include one or more of: 1) evoking, 2) partnering, 3) guiding, 4) expressing empathy, 5) strategic response to sustain talk, 6) strategic response to change talk, 7) reflection to question ratio, and 8) supporting autonomy and activation.
Aspect 24. The method of any of aspects 14-23, further comprising generating, via the one or more processors, a therapy recommendation for a subsequent therapy session based on the determined values of the one or more effectiveness parameters.
Aspect 25. The method of any of aspects 14-24, further comprising generating, via the one or more processors, a performance score of a practitioner from the cognitive or behavioral intervention session, based on the determined values of the one or more effectiveness parameters.
Aspect 26. The method of any of aspects 14-25, further comprising generating, via the one or more processors, a recommendation of a practitioner for a client based on the generated performance score of the practitioner.
Aspect 27. A computer-readable medium having stored thereon instructions that, when executed by a computing system comprising a processor, cause the computing system to perform operations comprising: obtaining a trained machine learning model trained to determine respective values of one or more effectiveness parameters of cognitive or behavioral intervention sessions, based on labeled training data indicative of respective values of the one or more effectiveness parameters for reference cognitive or behavioral intervention sessions; obtaining a transcript of a cognitive or behavioral intervention session; analyzing the transcript using the trained machine learning model to determine respective values of the one or more effectiveness parameters for the cognitive or behavioral intervention session; and synthesizing the determined respective values of the one or more effectiveness parameters for the cognitive or behavioral intervention session to generate an assessment outcome, wherein the assessment outcome includes a composite effectiveness score.
Aspect 28. The computer-readable medium of aspect 27, wherein the trained machine learning model includes one or more of: 1) one or more scoring models, or 2) one or more large language machine learning models.
Aspect 29. The computer-readable medium of any of aspects 27-28, wherein the operations further comprise utilizing a speaker diarization process to identify and separate respective contributions from a plurality of different speakers indicated by the transcript.
Aspect 30. The computer-readable medium of any of aspects 27-29, wherein the cognitive or behavioral intervention session is a motivational interview therapy session.
Aspect 31. The computer-readable medium of any of aspects 27-30, wherein the speaker diarization process includes identifying each respective one of the plurality of speakers as either an interviewer or an interviewee.
Aspect 32. The computer-readable medium of any of aspects 27-31, wherein the operations further comprise anonymizing audio data before analyzing using the trained machine learning model.
Aspect 33. The computer-readable medium of any of aspects 27-32, wherein the operations further comprise updating the trained machine learning model based on the determined respective values of the one or more effectiveness parameters for the cognitive or behavioral intervention session.
Aspect 34. The computer-readable medium of any of aspects 27-33, wherein the operations further comprise generating a report based on the respective values of the one or more effectiveness parameters for the cognitive or behavioral intervention session.
Aspect 35. The computer-readable medium of any of aspects 27-34, wherein the one or more effectiveness parameters include one or more of: 1) communication techniques, 2) engagement strategies, or 3) interaction skills.
Aspect 36. The computer-readable medium of any of aspects 27-35, wherein the one or more effectiveness parameters are further comprised of one or more effectiveness sub-parameters, wherein the one or more effectiveness sub-parameters include one or more of: 1) evoking, 2) partnering, 3) guiding, 4) expressing empathy, 5) strategic response to sustain talk, 6) strategic response to change talk, 7) reflection to question ratio, and 8) supporting autonomy and activation.
Aspect 37. The computer-readable medium of any of aspects 27-36, wherein the operations further comprise generating a therapy recommendation for a subsequent therapy session based on the determined values of the one or more effectiveness parameters.
Aspect 38. The computer-readable medium of any of aspects 27-37, wherein the operations further comprise generating a performance score of a practitioner from the cognitive or behavioral intervention session, based on the determined values of the one or more effectiveness parameters.
Aspect 39. The computer-readable medium of any of aspects 27-38, wherein the operations further comprise generating a recommendation of a practitioner for a client based on the generated performance score of the practitioner.
Aspect 40. Any one of aspects 1-39, in combination with any other suitable one of aspects 1-39.

### ADDITIONAL CONSIDERATIONS

The following considerations also apply to the foregoing discussion. Throughout this specification, plural instances may implement operations or structures described as a single instance. Although individual operations of one or more methods are illustrated and described as separate operations, one or more of the individual operations may be performed concurrently, and nothing requires that the operations be performed in the order illustrated. These and other variations, modifications, additions, and improvements fall within the scope of the subject matter herein.

It should also be understood that, unless a term is expressly defined in this patent using the sentence “As used herein, the term” “is hereby defined to mean . . . ” or a similar sentence, there is no intent to limit the meaning of that term, either expressly or by implication, beyond its plain or ordinary meaning, and such term should not be interpreted to be limited in scope based on any statement made in any section of this patent other than the language of the claims. To the extent that any term recited in the claims at the end of this patent is referred to in this patent in a manner consistent with a single meaning, that is done for sake of clarity only so as to not confuse the reader, and it is not intended that such claim term be limited, by implication or otherwise, to that single meaning. Finally, unless a claim element is defined by reciting the word “means” and a function without the recital of any structure, it is not intended that the scope of any claim element be interpreted based on the application of 35 U.S.C. § 112f.

Unless specifically stated otherwise, discussions herein using words such as “processing,” “computing,” “calculating,” “determining,” “presenting,” “displaying,” or the like may refer to actions or processes of a machine e.g., a computer that manipulates or transforms data represented as physical e.g., electronic, magnetic, or optical quantities within one or more memories e.g., volatile memory, non-volatile memory, or a combination thereof, registers, or other machine components that receive, store, transmit, or display information.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true or present and B is false or not present, A is false or not present and B is true or present, and both A and B are true or present.

In addition, use of “a” or “an” is employed to describe elements and components of the embodiments herein. This is done merely for convenience and to give a general sense of the invention. This description should be read to include one or at least one and the singular also includes the plural unless it is obvious that it is meant otherwise.

Upon reading this disclosure, those of skill in the art will appreciate still additional alternative structural and functional designs for implementing the concepts disclosed herein, through the principles disclosed herein. Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

1. A computing system comprising: a processor and a memory having stored thereon computer-executable instructions that, when executed, cause the computing system to:
obtain a trained machine learning model trained to determine respective values of one or more effectiveness parameters of cognitive or behavioral intervention sessions, based on labeled training data indicative of respective values of the one or more effectiveness parameters for reference cognitive or behavioral intervention sessions;
obtain a transcript of a cognitive or behavioral intervention session;
analyze the transcript using the trained machine learning model to determine respective values of the one or more effectiveness parameters for the cognitive or behavioral intervention session; and
synthesize the determined respective values of the one or more effectiveness parameters for the cognitive or behavioral intervention session to generate an assessment outcome, wherein the assessment outcome includes a composite effectiveness score.

2. The computing system of claim 1, wherein the trained machine learning model includes one or more of: 1) one or more scoring models, or 2) one or more large language machine learning models.

3. The computing system of claim 1, further comprising instructions that, when executed, cause the computing system to utilize a speaker diarization process to identify and separate respective contributions from a plurality of different speakers indicated by the transcript, wherein the speaker diarization process includes identifying each respective one of the plurality of speakers as either an interviewer or an interviewee.

4. The computing system of claim 1, wherein:
the cognitive or behavioral intervention session is a motivational interview therapy session; and
the transcript includes audio data; and
the computing system further comprises computer executable instructions that, when executed, cause the computing system to anonymize the audio data before analyzing using the trained machine learning model.

5. (canceled)

6. (canceled)

7. The computing system of claim 1, further comprising instructions that, when executed, cause the computing system to:
update the trained machine learning model based on the determined respective values of the one or more effectiveness parameters for the cognitive or behavioral intervention session; and
generate a report based on the respective values of the one or more effectiveness parameters for the cognitive or behavioral intervention session.

8. (canceled)

9. The computing system of claim 1, wherein: the one or more effectiveness parameters include one or more of: 1) communication techniques, 2) engagement strategies, or 3) interaction skills; and
the one or more effectiveness parameters are further comprised of one or more effectiveness sub-parameters, wherein the one or more effectiveness sub-parameters include one or more of: 1) evoking, 2) partnering, 3) guiding, 4) expressing empathy, 5) strategic response to sustain talk, 6) strategic response to change talk, 7) reflection to question ratio, and 8) supporting autonomy and activation.

10. (canceled)

11. The computing system of claim 1, wherein the computer-executable instructions, when executed, further cause the computing system to:
generate a therapy recommendation for a subsequent therapy session based on the determined values of the one or more effectiveness parameters;
generate a performance score of a practitioner from the cognitive or behavioral intervention session, based on the determined values of the one or more effectiveness parameters; and
generate a recommendation of a practitioner for a client based on the generated performance score of the practitioner.

12. (canceled)

13. (canceled)

14. A method comprising:
obtaining, via one or more processors, a trained machine learning model trained to determine respective values of one or more effectiveness parameters of cognitive or behavioral intervention sessions, based on labeled training data indicative of respective values of the one or more effectiveness parameters for reference cognitive or behavioral intervention sessions;
obtaining, via the one or more processors, a transcript of a cognitive or behavioral intervention session;
analyzing, via the one or more processors, the transcript using the trained machine learning model to determine respective values of the one or more effectiveness parameters for the cognitive or behavioral intervention session; and
synthesizing, via the one or more processors, the determined respective values of the one or more effectiveness parameters for the cognitive or behavioral intervention session to generate an assessment outcome, wherein the assessment outcome includes a composite effectiveness score.

15. The method of claim 14, wherein the trained machine learning model includes one or more of: 1) one or more scoring models, or 2) one or more large language machine learning models.

16. The method of claim 14, further comprising utilizing, via the one or more processors, a speaker diarization process to identify and separate respective contributions from a plurality of different speakers indicated by the transcript, wherein the speaker diarization process includes identifying each respective one of the plurality of speakers as either an interviewer or an interviewee.

17. The method of claim 14, wherein:
the cognitive or behavioral intervention session is a motivational interview therapy session; and
the transcript includes audio data; and
the method further comprises anonymizing, via the one or more processors, the audio data before analyzing using the trained machine learning model.

18. (canceled)

19. (canceled)

20. The method of claim 14, further comprising:
updating, via the one or more processors, the trained machine learning model based on the determined respective values of the one or more effectiveness parameters for the cognitive or behavioral intervention session; and
generating, via the one or more processors, a report based on the respective values of the one or more effectiveness parameters for the cognitive or behavioral intervention session.

21. (canceled)

22. The method of claim 14, wherein: the one or more effectiveness parameters include one or more of: 1) communication techniques, 2) engagement strategies, or 3) interaction skills, and
the one or more effectiveness parameters are further comprised of one or more effectiveness sub-parameters, wherein the one or more effectiveness sub-parameters include one or more of: 1) evoking, 2) partnering, 3) guiding, 4) expressing empathy, 5) strategic response to sustain talk, 6) strategic response to change talk, 7) reflection to question ratio, and 8) supporting autonomy and activation.

23. (canceled)

24. The method of claim 14, further comprising:
generating, via the one or more processors, a therapy recommendation for a subsequent therapy session based on the determined values of the one or more effectiveness parameters;
generating, via the one or more processors, a performance score of a practitioner from the cognitive or behavioral intervention session, based on the determined values of the one or more effectiveness parameters; and
generating, via the one or more processors, a recommendation of a practitioner for a client based on the generated performance score of the practitioner.

25. (canceled)

26. (canceled)

27. A computer-readable medium having stored thereon instructions that, when executed by a computing system comprising a processor, cause the computing system to perform operations comprising:
obtaining a trained machine learning model trained to determine respective values of one or more effectiveness parameters of cognitive or behavioral intervention sessions, based on labeled training data indicative of respective values of the one or more effectiveness parameters for reference cognitive or behavioral intervention sessions;
obtaining a transcript of a cognitive or behavioral intervention session;
analyzing the transcript using the trained machine learning model to determine respective values of the one or more effectiveness parameters for the cognitive or behavioral intervention session; and
synthesizing the determined respective values of the one or more effectiveness parameters for the cognitive or behavioral intervention session to generate an assessment outcome, wherein the assessment outcome includes a composite effectiveness score.

28. The computer-readable medium of claim 27, wherein the trained machine learning model includes one or more of: 1) one or more scoring models, or 2) one or more large language machine learning models.

29. The computer-readable medium of claim 27, wherein the operations further comprise utilizing a speaker diarization process to identify and separate respective contributions from a plurality of different speakers indicated by the transcript, wherein the speaker diarization process includes identifying each respective one of the plurality of speakers as either an interviewer or an interviewee.

30. The computer-readable medium of claim 27, wherein:
the cognitive or behavioral intervention session is a motivational interview therapy session;
the transcript includes audio data; and
the operations further comprise anonymizing the audio data before analyzing using the trained machine learning model.

31. (canceled)

32. (canceled)

33. (canceled)

34. (canceled)

35. The computer-readable medium of claim 27, wherein: the one or more effectiveness parameters include one or more of: 1) communication techniques, 2) engagement strategies, or 3) interaction skills; and
the one or more effectiveness parameters are further comprised of one or more effectiveness sub-parameters, wherein the one or more effectiveness sub-parameters include one or more of: 1) evoking, 2) partnering, 3) guiding, 4) expressing empathy, 5) strategic response to sustain talk, 6) strategic response to change talk, 7) reflection to question ratio, and 8) supporting autonomy and activation.

36. (canceled)

37. (canceled)

38. The computer-readable medium of claim 27, wherein the operations further comprise:
updating the trained machine learning model based on the determined respective values of the one or more effectiveness parameters for the cognitive or behavioral intervention session;
generating a report based on the respective values of the one or more effectiveness parameters for the cognitive or behavioral intervention session;
generating a therapy recommendation for a subsequent therapy session based on the determined values of the one or more effectiveness parameters;
generating a performance score of a practitioner from the cognitive or behavioral intervention session, based on the determined values of the one or more effectiveness parameters; and
generating a recommendation of a practitioner for a client based on the generated performance score of the practitioner.

39. (canceled)
